(19) United States
(12) Patent Application Publication
(10) Pub. No.: US 2013/0150617 A1
(43) Pub. Date: Jun. 13, 2013

(54) MONOLITHIC STRUCTURED CATALYST FOR CARBON MONOXIDE GASE-PHASE COUPLING TO DIALKYL OXALATE & PREPARATION METHOD AND APPLICATION THEREOF

(71) Applicants: Xinbin Ma, Tianjin (CN); Yujun Zhao, Tianjin (CN); Baowei Wang, Tianjin (CN); Shengping Wang, Tianjin (CN); Jing Lv, Tianjin (CN); Zhenghua Li, Tianjin (CN)

(72) Inventors: Xinbin Ma, Tianjin (CN); Yujun Zhao, Tianjin (CN); Baowei Wang, Tianjin (CN); Shengping Wang, Tianjin (CN); Jing Lv, Tianjin (CN); Zhenghua Li, Tianjin (CN)

(21) Appl. No.: 13/701,508

(22) PCT Filed: May 31, 2011
(86) PCT No.: PCT/CN2011/075018

§ 371 Date: Jan. 13, 2013

(30) Foreign Application Priority Data
Jun. 4, 2010 (CN) 201010191579.6
Jun. 4, 2010 (CN) 201010191580.9

(51) Int. Cl. B01J 29/04; C07C 67/36; B01J 23/89

(52) U.S. Cl. CPC B01J 29/04; B01J 23/8906; B01J 23/8986; B01J 23/8946; B01J 23/892; C07C 67/36
USPC 560/190; 502/326; 502/327; 502/261; 502/324; 502/74

(57) ABSTRACT

Provided is a monolithic catalyst for synthesizing an oxalate by carbon monoxide (CO) gaseous-phase coupling, a preparation method and the use thereof. In the catalyst, a ceramic honeycomb or a metal honeycomb was used as skeletal carrier, metal oxides were used as a carrier coating, precious metals Pt, Pd, Ir, Rh were used as active ingredients, as well as Fe, Co, Ni were used as additives, wherein the carrier coating accounts for 5 to 50 wt % of the honeycomb carrier the active ingredients of the catalyst account for 0.1 to 5 wt. % of the carrier coating; the additives of the catalyst account for 0.3 to 10 wt. % of the carrier coating; and the atomic ratio of the active ingredients to the additives was 0.1 to 3. the reaction for synthesizing the oxalate was carried out in a fixed bed reactor, wherein, N2 was used as a carrier gas. The volume ratio of N2:CO: Alkyl nitrite was 20-80:5-60:10-40, and the retention time was 0.5-10 s.

### REFERENCE TO PENDING APPLICATIONS

This application claims the benefit of International Application PCT/CN2011/075018 filed on May 31, 2011.

### REFERENCE TO MICROFICHE APPENDIX

This application is not referenced in any microfiche appendix.

### BACKGROUND OF INVENTION

This invention involves the dialkyl oxalate syntheses, especially a monolithic catalyst and its preparation for carbon monoxide gas-phase coupling to dialky oxalate, and the production process of carbon monoxide gas-phase coupling to dialky oxalate catalyzed by the monolithic catalyst, producing method of dialkyl oxalate by CO gaseous phase coupling using this monolithic catalyst thereof.

Nowadays, with the petroleum resource of the world increasingly exhausted, the technologies for synthesizing ethylene glycol(EG) from coal or natural gas material have a very important practical significance. And the technology was widely acknowledged as a significant Cl chemical technology owing to its scientific raw material route and reasonable resource utilization. Dialkyl oxalate was considered as a crucial intermediate product in synthesizing EG, and the synthetic technology of dialkyl oxalate was one of the core technologies in synthesizing EG from coal or natural gas. Besides, dialkyl oxalate was also crucial organic chemical materials, which was widely used in fee chemical engineering to produce all sorts of dyes, medicine, important solvents, extractants, and various kinds of intermediate products.

There were many disadvantages in traditional technology of dialkyl oxalate production, such as high cost, huge energy consumption, serious pollution and unreasonable raw material utilization. At present, one relatively advanced method was alcohol oxidization carbonylation, especially la the system of CO gas-phase coupling to dialky oxalate, we introduce nitrous acid ester(RONO, R for alkyl) as oxygen carrier during gaseous alcohol oxidization carbonylation, making the reaction conducting under mild condition. Nitric oxide generated during reaction process can react with alcohol and oxygen to form alkyl nitrite, thus the whole technology became a self-sealing and cyclic system without the three wastes. The reaction equations were described as follows:

Coupling reaction: 2RONO+2CO→(COOR)2+2NO

Regeneration reaction: 2NO+2ROH+1/2O2→2RONO+H2O

This method owns many advantages such as extensive raw material source, fine atom economy, moderate reaction condition, less energy consumption, pollution-free process, high product selectivity and quality. Due to its clean production technology, obvious economic and social benefits, the process has attracted widespread concern all over the world. At present, this process was under research or industrial development stage. Some advancement has been acquired in catalyst preparation, activity, support effect aid operation conditions. Nevertheless, noble metal palladium was generally used as active component of catalysts, which was expensive and increased fee production cost of dialkyl oxalate, reducing the economic efficiency of fee technology route consequently.

Monolithic structured catalyst has neat parallel longitudinal channels, lower pressure drop and benefit the operation at higher space velocity. It was characterized by small reactor volume, whole assembly, easy replacement, fine mass-transfer effect, low loading of active component and high activity. The application of the monolithic structured catalysts was gained more and more attention in gas-solid or gas-liquid-solid heterogeneous reaction in recent years. However, no literature about monolithic structured catalyst research has been reported with regard to dialkyl oxalate production from CO gaseous phase coupling.

### SUMMARY OF THE INVENTION

One purpose of this invention is provided a kind of monolithic catalyst for carbon monoxide gas-phase coupling to dialky oxalate. This kind of monolithic catalysts reduced dosage of precious metal and had high catalytic activity as well as low cost. It provided a new route for dialkyl oxalate production from coal or natural gas, which can greatly promote the industrialization for the technology of carbon monoxide gas-phase coupling to dialkyl oxalate.

Another purpose of this invention is to offer a preparation method of the monolithic catalyst for carbon monoxide gas-phase coupling to dialkyl oxalate. Use dip-coating method to load washcoat on cordierite ceramic honeycomb support, alkaline solution and the H2 or CO atmosphere were introduced to treat the coated honeycomb support, then, the catalytic activity can be elevated. Meanwhile, active components of this catalyst were confined to the washcoat. The coating was so thin that diffusion resistance was reduced and the mass-transfer efficiency of reactants between gas-solid or gas-liquid-solid phase was elevated, the contact area between reactants and catalyst was also enlarged. Thus the catalytic ability of active components was enhanced.

This invention also aimed at providing a method for carbon monoxide gas-phase coupling to dialkyl oxalate using the monolithic catalyst. The use of the monolithic catalyst rather than traditional pellet catalyst can reduce the pressure drop of the catalyst bed and promote the production capability of dialkyl oxalate for single set of equipment. At the same time, the depletion of the catalyst, resulting from the abrasion during packing process and reaction process, can also be minimized so that the use-cost of the catalyst can be reduced. This invention could be able to realize large-scale engineering application due to its high catalytic activity, low cost, easy replacement, etc.

### BRIEF DESCRIPTION OF THE DRAWINGS

FIG. 1 was the appearance map of the honeycomb ceramics and the monolithic catalyst coating with active components, the ordered parallel channel structure can be observed.

FIG. 2 presented the structure diagrams of single pore in the monolithic catalyst.

FIG. 3 showed the SEM image of single-wall: structure of the monolithic catalyst.

FIG. 4 showed the distribution map of the elements from the single-walled cross-section of monolithic catalyst.

FIG. 5 showed the stability date of the monolithic catalyst for synthesizing dialkyl oxalate by CO gas-phase coupling before and after the modification with additive.

### DESCRIPTION OF THE INVENTION

Provided was the monolithic structured catalyst for carbon monoxide gas-phase coupling to dialkyl oxalate. In the catalyst, a ceramic honeycomb or a metal honeycomb was used as skeletal carrier, metal oxides were used as a carrier coating, precious metals Pt, Pd, Ir, Eh were used as active components, as well as Fe, Co, Ni were used as additives.

Specific preparation procedure of the catalyst was as follows;

Metal nitrate, hydroxide or oxide were blended with dilute nitric acid; then the mixture was bail milled in a ball mill equipment to prepare hail-milling slurry for coating the support; the washcoat was loaded on ceramic or metal honeycomb supports use dip-coating method with ball-milling slurry; the washcoated support was dried and calcined in a muffle furnace to form metal oxide washcoat then the washcoated support calcined was impregnated in the precursor solutions of active components and additive to load active components and additive; the impregnated support was dried and finally treated in H2 or CO atmosphere.

In this invention, the components of the metal oxide washcoat were selected from the following oxides: Al2O3, SiO2, ZrO2 TiO2, Fe2O3, La2O3, CuO, ZnO, Cr2O3, GaO, BaO; CaO, MgO, MnO, Or

In this invention, the components of the metal oxide washcoat were selected from the following oxides: Al2O3, SiO2, ZrO2, TiO2, Fe2O3, La2O3, CuO, ZnO, Cr2O3, GaO, BaO, CaO.

In this invention, the active ingredient was selected from the precious metal Pt, Pd, Ir, Rh and mixtures thereof.

In this invention, the additive of the monolithic structured catalyst for synthesizing an oxalate by carbon monoxide(CO) gaseous-phase coupling was selected from the Fe, Co, Ni and mixtures thereof.

In this invention, the additive of the monolithic structured catalyst for synthesizing an oxalate by carbon monoxide(CO) gaseous-phase coupling also includes Cu or Ce.

In this invention for the monolithic structured catalyst, the washcoat on the carrier accounts for 5 to 50 wt. % of the honeycomb carrier; me active Ingredients of the catalyst account for 0.1 to 5 wt. % of the washcoat on the carder; the additives of the catalyst account for 0.03 to 10 wt % of the washcoat on the carrier; and the atomic ratio of the active ingredients to the additives was 0.01 to 5.

In this invention for the monolithic structured catalyst, the washcoat on the carrier accounts for 5 to 50 wt % of the honeycomb carrier; the active ingredients of the catalyst account for 0.1 to 5 wt % of the washcoat on the carrier; the additives of the catalyst account for 0.3 to 10 wt. % of the washcoat on the carrier; and the atomic ratio of the active ingredients to the additives was 0.1 to 5.

In this invention for the monolithic structured catalyst, the washcoat on the carrier accounts for 5 to 30 wt. % of the honeycomb carrier; the active ingredients of the catalyst account for 0.1 to 2 wt. % of the washcoat on the carrier; the additives of the catalyst account for 0.3 to 6 wt. % of the washcoat on the carrier; and the atomic ratio of the active ingredients to the additives was 0.1 to 3.

This invention provided the preparation method of monolithic structured catalyst for carbon monoxide gas-phase coupling to dialkyl oxalate. It was characterized that the monolithic catalyst preparation method comprising the steps of:

1) Preparation of the ball milling slurry; Mix one or various kinds of metal nitrate, hydroxide or oxide; add dilute nitric acid Into the mixture; then adjust the pH of the mixture in the range of 1 to 4; finally the mixture was ball milled for 1 to 48 hours to get the ball milling slurry for the coating of the supports.

2) Washcoat loading: The washcoat was loaded on ceramic or metal honeycomb supports use dip-coating method with ball-milling slurry, followed by a drying process; one or multiple dip-coating was performed to meet the standard loading; finally calcination was performed at 900-1300° C. for 1-12 hours to form washcoat.

3) Active components and additive loading; Coated support was impregnated into precursor solutions with one or various active components and additives, after a drying process, the catalyst was treated with H2 or CO atmosphere for 1-10 hours to get the very catalyst we wanted.

This invention provides the preparation method of monolithic catalyst for the reaction of CO gaseous phase coupling to dialkyl oxalate. The specific procedures were as follows:

1) Preparation of the ball milling slurry: Mix one or various kinds of metal nitrate, hydroxide or oxide; add dilute nitric acid of 1-15 wt % into the mixture; then adjust the pH of the mixture in the range of 1 to 4; finally the mixture was ball milled for 3 to 20 hours to get the ball milling slurry for the coating of the supports.

2) Washcoat loading: The washcoat was loaded on cordierite ceramic honeycomb support or metal honeycomb support use dip-coating method with ball-milling slurry, followed by drying at 70-130° C. for 2-4 hours, the coated support was calcined in a furnace at 900-1200° C. for 1-12 hours to form washcoat, the washcoat loading accounts for 5-50 wt. % of honeycomb support, multiple dip-coating must he performed to get higher washcoat loading.

3) Active components loading: Coated support was impregnated into precursor solutions with one or various active components and additives for 3 minutes to 12 hours to load the active components and additives, then the impregnated honeycomb support was drying at 70-130° C. for 1-12 hours, finally, the catalyst was treated with H2 or CO atmosphere at 400-800° C. for 1-10 hours to get the very catalyst we wanted.

In the above procedure 3, the impregnated washcoat with active components and additive should be dipped in the 0.01-2M alkaline solution for 0.5-24 hours after drying.

The alkaline solution was selected from NaOH, KOH, Na2CO3, K2CO3, NaHCO3, KHCO3 and mixtures thereof.

The precursors of active components used in procedure 3 was selected from palladium chloride, palladium bromide, platinum chloride, rhodium chloride, palladium nitrate, platinum nitrate, palladium acetate, rhodium acetate. Palladium chloride and palladium acetate were preferred. Salts of platinum group can be used singly or in combination.

The precursors of additive used in procedure 3 were selected from ferric trichloride, cobalt bromide, ferric nitrate, nickel nitrate, cobalt acetate, nickel acetate and mixtures thereof. The monolithic catalyst of this Invention was used in reaction of CO gaseous coupling to dialkyl oxalate which can be dimethyl oxalate or diethyl oxalate or mixtures thereof.

This invention provides the production method of dialkyl oxalate by CO gaseous phase coupling using the very monolithic catalyst. The method includes the following steps: the reaction for synthesizing the oxalate was carried out in a fixed bed reactor, the catalyst bed was filled with the monolithic structured monolithic catalyst supporting noble metal, the react pressure was 0.1-2 MPa, the reaction temperature was 80-200° C., N2 was used as carrier gas. CO and gasified alkyl nitrite were introduced into the reactor and react on the monolithic structured catalyst to produce dialkyl oxalate, The volume ratio of N2:CO:Alkyl nitrite was 20-80:5-60:10-40, and the retention time was 0.5-10 s.

The method in this invention, the catalyst bed was filled by a monolithic catalyst loaded with precious metal.

The method in this invention, the reaction pressure was 0.1-1.2 MPa and the reaction temperature was 90-150° C.

The method in this invention, the volume ratio of the feed gas was: N2:CO:alkyl nitrite to be 20-80:5-60:10-40. The retention time was 1-10 s.

The method in this invention, the volume ratio of the feed gas was: N2:CO: alkyl nitrite is to be 20-80:5-60:5-10.

The method in this invention, alkyl nitrite was selected from methyl nitrite or ethyl nitrite or both of them.

Compared with general technology, this invention includes the following characteristics;

1. The monolithic catalyst in this invention was applied to dialkyl oxalate production from CO gaseous coupling for the first time, giving rise to a brand new idea for developing catalyst used for dialkyl oxalate production from CO gaseous coupling.

2. In the monolithic catalyst preparation. Alkali treatment was introduced to improve the interaction between active components and supports, which effectively enhanced catalytic activity in the reaction of CO coupling to dialkyl oxalate.

3. in the synthesis of dialkyl oxalate by carbon monoxide (CO) gas-phase coupling

Compared with pellet catalyst, the monolithic catalyst in this invention greatly reduces the internal diffusion resistance, because the active components of the catalyst were mainly concentrated on the ultra-thin washcoat. Consequently, this monolithic catalyst elevated the mass-transfer efficiency of reaction stuff between gas and solid phase and reduced the amount of precious metals (far less than conventional pellet catalyst for at least 86%). Therefore, catalyst cost can be vastly reduced without affecting reaction activity. Thus, economical efficiency was greatly improved in dialkyl oxalate production from CO gas-phase coupling.

4. Compared with pellet catalyst in the reaction of CO gaseous coupling to dialkyl oxalate, this monolithic catalyst can reduce the pressure drop of the catalyst bed and decreased energy depletion. Moreover, it was suitable for the reaction in the catalyst bed with higher height-diameter ratio. Thus, the production ability of dialkyl oxalate for single equipment can be substantially enhanced and the operation cost can be reduced.

Hence, in this invention, the monolithic structured catalyst used for the coupling of CO to dialkyl oxalate possessed both novelty and economic efficiency. It provide a new technology route for synthesizing dialkyl oxalate from coal or natural gas and promote engineering realizing of dialkyl oxalate production from CO coupling.

The following examples illustrate the present Invention more specifically.

### EXAMPLE 1

Preparation of the Wash Coating Slurry

12.5 grams of γ-Al2O3, 3.5 grams of AlOOH, 6.5 grams of Al(OH)3, 8.0 grams of Al(NO3)3 and 100 ml 10 wt. % dilute nitric acid were weighted and mixed together, then the mixed material was ball milled at the rotating speed of 200 rpm for 16 hours to get the alumina slurry, ( planetary ball mill XQM-2L, NanJingShunChi for use )

Preparation of the Catalyst

The cordierite ceramic honeycomb with 400 cells per square inch (Φ25 mm×25 mm) was calcined at 700° C. for 2 hours to remove the organic impurities, then alumina washcoat was loaded via the conventional dip-coating method with the above alumina slurry. Then the resulting coated honeycomb was dried in microwave and weighted. Several times dip-coating were performed until the Al2O3 washcoat loading reach 20%. The coated support was calcined in a furnace with the temperature raised to 1200° C. and maintain at this temperature for 4 hours. Then, the coated support was impregnated in PdCl2-FeCl3 solutions for 5 minutes, the concentrations of PdCl2 and FeCl3 in the solution were 0.2M and 13M, respectively. The impregnated catalyst was dried, followed by a treatment in H2 at 500° C. for 4 hours. The obtained catalyst can be denoted as 1.0% Pd—Fe/20% α-Al2O3/Cordierite (atomic ratio of Pd/Fe was 1.5:1) with Pd content of 1.0 wt. % (relative to Al2O3 washcoat) and Pd/Fe atomic ratio of 1.5:1.

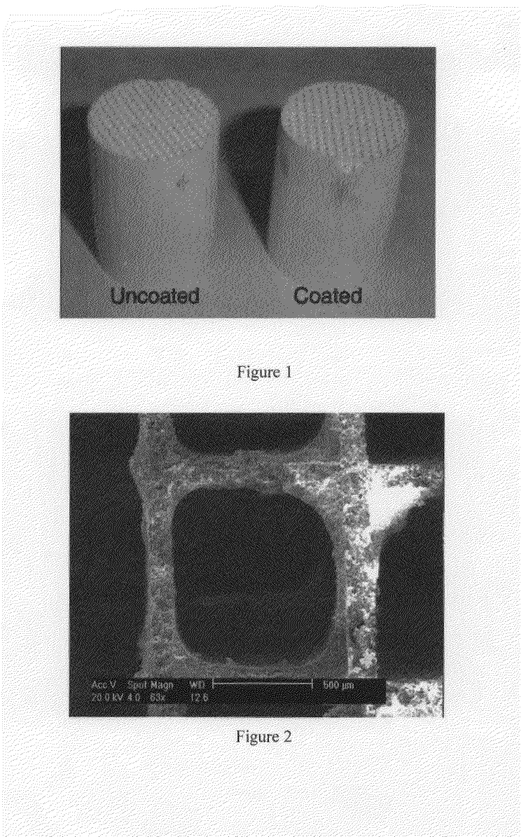

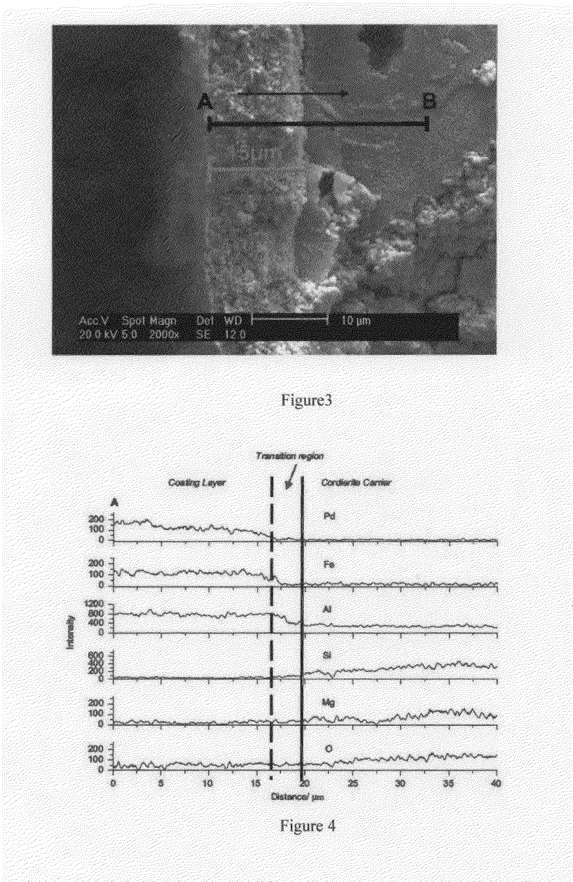

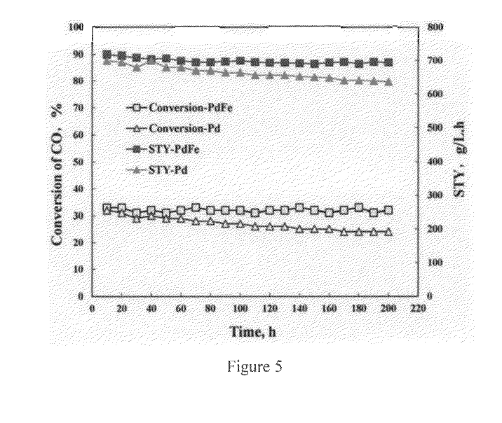

FIG. 1 showed the appearance map of the honeycomb ceramics and the monolithic catalyst loaded with washcoat and active components, where the ordered parallel channel structure can be observed. FIG. 2 presented the structure diagrams of single pore in the monolithic catalyst. FIG. 3 showed the SEM image of single-wall structure of the monolithic catalyst, where the Al2O3 washcoat was found mainly attached at the outside surface of honeycomb substrate. FIG. 4 showed the distribution map of the elements from the single-walled cross-section of monolithic catalyst (scanning from the orientation of A to B shown in FIG. 3). Seen from the distribution of Al element, it was found that the Al2O3 washcoat mainly focus at the outside surface of honeycomb substrate with thickness of 15 μm. While the active component Pd was evenly distributed in the washcoat layer, and rarely entered into the honeycomb substrate, presenting an egg-shell like distribution which may greatly reduce the internal diffusion resistance significantly.

Synthesis Method of Oxalate

The above prepared monolithic catalyst of 12 ml was installed into a fixed bed reactor, after purging the system with nitrogen, CO and methyl nitrite were preheated and introduced into the system to synthesis dimethyl oxalate on the monolithic catalyst. The reaction was carried out at 110° C. and 0.1 MPa. The feed volume ratio of N2:CO: methyl nitrite was kept at 50:30:20 and residence time was 1.5 seconds. Results of the reaction were shown in table 1.

### EXAMPLE 2

12 ml of the catalyst prepared according to Example 1 was installed into a fixed bed reactor, after purging the system with nitrogen, CO and methyl nitrite were preheated and introduced into the system to synthesis dimethyl oxalate on the monolithic catalyst. The reaction was carried out at 120° C. and 0.25 MPa. The feed volume ratio of N2:CO; methyl nitrite was kept at 50:30:20 and residence time was 7.5 seconds. The reaction was performed for 200 hours. Results of the reaction were shown in FIG. 5. As can be seen from FIG. 5, the monolithic catalyst modified with additive presented much higher stability.

### COMPARATIVE EXAMPLE 1

Preparation of Catalysts

The A catalyst was prepared in the same manner as Example 1, except using PdCl2 solution of 0.2M in place of the mixed solution of PdCl2 and FeCl3 during the impregnation of the active components. The obtained catalyst can he denoted as 1.0% Pd/20% α-Al2O3/Cordterite with Pd content of 1.0 wt. % (relative to Al2O3 washcoat).

Oxalate was synthesized In the same manner as Example 2, and the results were listed in FIG. 5.

### EXAMPLE 3

The catalyst was prepared in the same manner as Example 1, except using the ceramic honeycomb of cordierite with 600 cells per square inch (Φ25 mm×25 mm) as the carrier. The obtained catalyst can be denoted as 1.0% Pd—Fe/20% α-Al2O3/Cordierite (atomic ratio of Pd/Fe was 1.5:1) with Pd content of 1.0 wt. % (relative to Al2O3 washcoat) and Pa/Fe atomic ratio of 1.5:1. Oxalate was synthesized in the same manner as Example 1, and the results were listed in Table 1.

### EXAMPLE 4

The catalyst was prepared in the same manner as Example 1, except the milling time was 4.5 hours. The obtained catalyst can be denoted as 1.0% Pd—Fe/20% α-Al2O3/Cordierite (atomic ratio of Pd/Fe was 1.5:1). Oxalate was synthesized in the same manner as Example 1, and the results were listed in Table 1.

### EXAMPLE 5

The catalyst was prepared in the same manner as Example 1, except the milling time was 9 hours. The obtained catalyst can be denoted as 1.0% Pd—Fe/20% α-Al2O3/Cordierife (atomic ratio of Pd/Fe was 1.5:1). Oxalate was synthesized in the same manner as Example 1, and the results were listed in Table 1.

### EXAMPLE 6

The catalyst was prepared in the same manner as Example 1, except the milling time was 36 hours. The obtained catalyst can be denoted as 1.0% Pd—Fe/20% α-Al2O3/Cordierite (atomic ratio of Pd/Fe was 1.5:1). Oxalate was synthesized in the same manner as Example 1, and the results were listed in Table 1.

### EXAMPLE 7

The catalyst was prepared in the same manner as Example 1, except diluting the slurry to 0.8 times to get the Al2O3 washcoat content of 5% and replacing the molar concentration of PdCl2 and FeCl3 with 0.2M and 0.1M respectively. The obtained catalyst can be denoted as 1.0% Pd—Fe/5% α-Al2O3/Cordierite (atomic ratio of Pd/Fe was 2:1) with Pd content of 1.0 wt. % (relative to Al2O3 washcoat) and Pa/Fe atomic ratio of 2:1. Oxalate was synthesized in the same manner as Example 1, and the results were listed in Table 1.

### EXAMPLE 8

The catalyst was prepared in the same manner as Example 1, except diluting the slurry to 0.8 times and multiple dip-coating to get the Al2O3 washcoat content of 10 wt. % and replacing the molar concentration of PdCl2 and FeCl3 with 0.2M and 0.1M respectively. The obtained catalyst can he denoted as 1.0% Pd—Fe/10% α-Al2O3/Cordierite (atomic ratio of Pd/Fe was 2:1) with Pd content of 1.0 wt % (relative to Al2O3 washcoat) and Pa/Fe atomic ratio of 2:1. Oxalate was synthesized in the same manner as Example 1, and the results were listed in Table 1.

### EXAMPLE 9

The catalyst was prepared in the same manner as Example 1, except increasing the dip-coating times in the ball milled slurry to get the Al2O3 washcoat content of 30 wt % and replacing the molar concentration of PdCl2 and FeCl3 with 0.2M and 0.067M respectively. The obtained catalyst can he denoted as 1.0% Pd—Fe/30% α-Al2O3/Cordierite (atomic ratio of Pd/Fe was 3:1) with Pd content of 1.0 wt % (relative to Al2O3 washcoat) and Pa/Fe atomic ratio of 3:1. Oxalate was synthesized In the same manner as Example 1, and the results were listed in Table 1.

### EXAMPLE 10

The cordierite ceramic honeycomb with 400 cells per square inch (Φ25 mm×25 mm) was calcined at 700° C. for 2 hours to remove the organic impurities. Then, the ceramic honeycomb carriers was soaked in the alkaline or acidic silica solution via the conventional dip-coating method, followed by drying in microwave to get silica washcoat on the support. Several times dip-coating were performed until the silica washcoat loading reach 20%. The coated support was calcined in a furnace with the temperature raised to 900° C. and maintain at this temperature for 4 hours. Then, the coated support was impregnated in PdCl2-FeCl3 solutions for 5 mutates, the concentrations of PdCl2 and FeCl3 in the solution were 0.2M and 0.13M, respectively. The impregnated catalyst was dried, followed by a treatment in H2 at 500° C. for 4 hours. The obtained catalyst can be denoted as 1.0% Pd—Fe/20% SiO2/Cordierite (atomic ratio of Pd/Fe was 1.5:1) with Pd content of 1.0 wt. % (relative to Al2O3 washcoat) and Pa/Fe atomic ratio of 1.5:1. Oxalate was synthesized in the same manner as Example 1, and the results were listed in Table 1.

### EXAMPLE 11

Preparation of Wash Coating Slurry

15.0 grams of met titanic acid were weighted and 10 mL hydrochloric acid and 10 ml nitricacid were added. The mixture was ball-milled for 16 hours to obtain titanium oxide slurry.

Preparation of Catalyst

The catalyst was prepared in the same manner as Example 8, except the titanium oxide slurry was used as the precursor of the washcoat The obtained catalyst can be denoted as 1.0% Pd—Fe/20% TiO2/Cormeriie (atomic ratio of Pd/Fe was 1.5:1).

Synthesis of Oxalate

Oxalate was synthesized in the same manner as Example 1, and the results were listed in Table 1.

### EXAMPLE 12

Preparation of Wash Coating Slurry

16.5 grams of Zr(OH)4, 15.0 grams of Zr(NO3)4.5H2O and 3.0 grams of ZrO2 wore weighted and 50 mL nitric acid was added. The mixture was ball-milled for 16 hours to obtain zirconium oxide slurry.

Preparation of Catalyst

The catalyst was prepared in the same manner as Example 8, except the zirconium oxide slurry was used as the precursor of the washcoat. The obtained catalyst can be denoted as 1.0% Pd—Fe/20% ZrO2/Cordierite (atomic ratio of Pd/Fe was 1.5:1).

Synthesis of Oxalate

Oxalate was synthesized in the same manner as Example 1, and the results were listed in Table 1.

### EXAMPLE 13

The catalyst was prepared in the same manner as Example 1, except 1.2 grams of Mg(NO3)2 was added into the wash coating slurry. The obtained catalyst can be denoted as 1.0% Pd—Fe/20% Al2O3-MgO/Cordierite (Pd/Fe atomic ratio of 1.5:1). Oxalate was synthesized in the same manner as Example 1, and the results were listed in Table 1.

### EXAMPLE 14

The catalyst was prepared in the same manner as Example 1, except 0.5 grams of Mn(NO3)2 was added into the wash coating slurry. The obtained catalyst can be denoted as 1.0% Pd—Fe/20% Al2O3-MnO/Cordierite (Pd/Fe atomic ratio of 1.5:1). Oxalate was synthesized in the same manner as Example 1, and the results were listed in Table 1.

### EXAMPLE 15

The catalyst was prepared in the same manner as Example 1, except changing the molar concentration of PdCl2 and FeCl3 to 0.02M and 0.013M to get a Pd loading of 0.1 wt. %. The obtained catalyst can be denoted as 0.1% Pd—Fe/20% α-Al2O3/Cordierite (atomic ratio of Pd/Fe was 1.5:1) with Pd content of 0.1 wt. % (relative to Al2O3 washcoat) and Pa/Fe atomic ratio of 1.5:1. Oxalate was synthesized in the same manner as Example 1, and the results were listed in Table 1.

### EXAMPLE 16

The catalyst was prepared in the same manner as Example 1, except changing the molar concentration of PdCl2 and FeCl3 to 0.4M and 0.27M to get a Pd loading of 2 wt %. The obtained catalyst can be denoted as 2% Pd—Fe/20% α-Al2O3/Cordieri.te (atomic ratio of Pd/Fe was 1.5:1) with Pd content of 2 wt. % (relative to Al2O3 washcost) and Pa/Fe atomic ratio of 1.5:1. Oxalate was synthesized in the same manner as Example 1, and the results were listed in Table 1.

### EXAMPLE 17

The catalyst was prepared in the same manner as Example 1, except changing the molar concentration of PdCl2 and FeCl3 to 0.2M and 2M. The obtained catalyst can be denoted as 1% Pd—Fe/20% α-Al2O3/Cordierke (atomic ratio of Pd/Fe was 0.1:1) with Pd content of 1.0 wt. % (relative to Al2O3 washcoat) and Pa/Fe atomic ratio of 0.1:1. Oxalate was synthesized in the same manner as Example 1, and the results were listed in Table 1.

### EXAMPLE 18

The catalyst was prepared in the same manner as Example 1, except changing the molar concentration of PdCl2 and FeCl3 to 0.2M and 0.08M. The obtained catalyst can be denoted as 1.0% Pd—Fe/20% α-Al2O3/Cordierite (atomic ratio of Pd/Fe was 2.5:1) with Pd cement of 1.0 wt. % (relative to Al2O3 washcoat) and Pa/Fe atomic ratio of 2.5:1. Oxalate was synthesized in the same manner as Example 1, and the results were listed in Table 1.

### EXAMPLE 19

The catalyst was prepared in the same manner as Example 1, except changing precursor solution of active component to be a hydrochloric add solution of Pt(NO3)2-Ni(NO3)2 (The concentrations of Pt(NO3)2 and Ni(NO3)2 were 0.02M and 0.02M, respectively). The obtained catalyst can be denoted as 1.0% Pd—Ni/20% α-Al2O3/Gordierite (atomic ratio of Pd/Ni was 1:1) with Pd content of 0.1 wt. % (relative to Al2O3 washcoat) and Pa/Ni atomic ratio of 1:1. Oxalate was synthesized in the same manner as Example 1, and the results were listed In Table 1.

### EXAMPLE 20

The catalyst was prepared in the same manner as Example 1, except using hydrochloric acid solution of PdCl2-IrCl4-FeCl3 solution Instead of PdCl2-FeCl3 (The concentrations of PdCl2, IrCl4 and FeCl3 were 0.15M, 0.03M and 0.13M, respectively). The obtained catalyst can be denoted as 0.8% Pd-0.1% Ir—Fe/20% α-Al2O3/Cordierlte ((Pd+Ir)/Fe atomic ratio of 1.2:1). Oxalate was synthesized In the same manner as Example 1, and the results were listed in Table 1.

### EXAMPLE 21

The catalyst was prepared in the same manner as Example 1, except a treatment by Na2CO3 solution of 0.2M was performed for 6 hours after the impregnation In the hydrochloric acid solution of PdCl2-FeCl3 and a subsequent drying process. The obtained catalyst can be denoted as 1.0% Pd—Fe/20% α-Al2O3/Cordierite (atomic ratio of Pd/Fe was 1.5:1) with Pd content of 1.0 wt. % (relative to Al2O3 washcoat) and Pd/Fe atomic ratio of 1.5:1. Oxalate was synthesized in the same manner as Example 1, and the results were listed in Table 1.

### EXAMPLE 22

The catalyst was prepared in the same manner as Example 21, except alkali treatment by NaOH solution instead of Na2CO3 solution. The obtained catalyst can be denoted as 1.0% Pd—Fe/20% α-Al2O3/Cordierite (atomic ratio of Pd/Pe was 1.5:1) with Pd content of 1.0 wt % (relative to Al2O3 washcoat) and Pd/Fe atomic ratio of 1.5:1. Oxalate was synthesized in the same manner as Example 1, and the results were listed in Table 1.

### EXAMPLE 23

The catalyst was prepared in the same manner as Example 1, except the reduction was performed at 200° C. for 10 hours using CO. The obtained catalyst can be denoted as 1.0% Pd—Fe/20% α-Al2O3/Cordierite (atomic ratio of Pd/Fe was 1.5:1} with Pd content of 1.0 wt. %. (Relative to Al2O3 washcoat) and Pd/Fe atomic ratio of 1.5:1. Oxalate was synthesized in the same manner as Example 1, and the results were listed in Table 1.

### EXAMPLE 24

Metal honeycomb support (Triangle channel, 400 cpsi, Φ25 mm×25 ) was washed in acetone and ethanol to remove the organic compounds on the surface of the support, followed by washing with deionized water and calculation at 800° C. for 10 hours. Then, the metal honeycomb coat treated was coated with Al2O3 ball-milled slurry of alumina as mentioned in Example 1 via the conventional dip coating method and dried in an oven. Several times dip-coating were performed until the Al2O3 washcoat loading reach 20%. The coated support was calcined in a furnace with the temperature raised to 1200° C. and maintain at this temperature for 4 hours. Then, the coated support was impregnated in a solution of PdCl2 and FeCl3, both concentrations of PdCl2 and FeCl3 were 0.2M. The impregnated catalyst was dried, followed by a treatment in H2 at 500° C. for 4 hours. The obtained catalyst can be denoted as 1.0% Pd—Fe/20% α-Al2O3/Metal monolith with Pd content of 1.0 wt. % (relative to Al2O3 washcoat) and Pd/Fe atomic ratio of 1:1.Oxalate was synthesized in the same manner as Example 1 except the use of metal honeycomb support, and the results were listed in Table 1.

### COMPARATIVE EXAMPLE 2

The catalyst was prepared in the same manner as Example 1, except the calcination was performed at 400° C. for 2 hours after impregnating active components and drying. The obtained catalyst can be denoted as 1.0% Pd—Fe/20% α-Al2O3/Cordierite (atomic ratio of Pd/Fe was 1.5:1) with Pd content of 1.0 wt. %. (Relative to Al2O3 washcoat) and Pd/Fe atomic ratio of 1.5:1. Oxalate was synthesized in the same manner as Example 1, and the results were listed in Table 1.

### COMPARATIVE EXAMPLE 3

Preparation of Wash Coating Slurry

γ-Al2O3 powders with 200 meshes was used as the raw material to prepare 20% γ-Al2O3 suspension, whose pH was adjusted to 5 with dilute nitric acid and the mixture was stirred for 24 hours to obtain alumina slurry for use.

Preparation of Catalyst

The catalyst was prepared in the same manner as Example 1, except the alumina slurry prepared in this example was used. The obtained catalyst can be denoted as 1.0% Pd—Fe/20% α-Al2O3/Cordierite (atomic ratio of Pd/Fe was 1.5:1). Oxalate was synthesized in the same manner as Example 1; the results were listed in Table 1.

### COMPARATIVE EXAMPLE 4

Granular type α-Al2O3 with diameter of Φ2-3 mm was used, which was calcined in a furnace with the temperature raised to 1200° C. and maintain at this temperature for 4 hours to obtained the particulate catalyst support. Them the support was impregnated in hydrochloric acid solution of PdCl2 and FeCl3 (concentrations of PdCl2 and FeCl3 were 0.02M and 0.013M). The impregnated catalyst was dried, followed by a reduction in H2 at 500° C. for 4 hours. The obtained catalyst can be denoted as 0.1% Pd—Fe/α-Al2O3 with Pd content of 0.1 wt % and Pd/Fe atomic ratio of 1.5:1. Oxalate was synthesized in the same manner as Example 1 except the use of particulate catalyst, and the results were listed in Table 1.

### COMPARATIVE EXAMPLE 5

The catalyst was prepared in the same manner as Comparative Example 5, except changing the molar concentration of PdCl2 and FeCl3 to 0.2M and 0.13M. The obtained catalyst can be denoted as 10% Pd—Fe/α-Al2O3 with Pd content of 1.0 wt. % (Relative to Al2O3 support) and Pa/Fe atomic ratio of 1.5:1. Oxalate was synthesized in the same manner as Example 1, and the results were listed in Table 1.

### EXAMPLE 25

The catalyst prepared in Example 1 was used for the synthesis of diethyl oxalate by CO gaseous coupling at the reaction conditions as follows: temperature at 120° C, pressure at 0.1 MPa, residence time of 1.5 seconds, feed volume ratio of N2:CO:C2H5ONO=40:40:20 and the catalyst dosage of 12 ml. The results were listed in Table 2.

### EXAMPLE 26

The catalyst prepared in Example 1 was used for the synthesis of diethyl oxalate by CO gaseous coupling at the reaction conditions as follows: temperature at 130° C., pressure at 0.3 MPa, residence time of 3 seconds, feed volume ratio of N2:CO:C2H5ONO=40:40:20 and the catalyst dosage of 48 ml. The results were listed In Table 2

### EXAMPLE 27

The same method as Example 1 was used except the pressure of 0.6 MPa and residence time of 3.6 seconds were adopted. The results were listed in Table 2.

### EXAMPLE 28

The same method as Example 1 was used except the reaction temperature of 90° C. was adopted. The results were listed in Table 2.

### EXAMPLE 29

The same method as Example 1 was used except the reaction temperature of 150° C. was adopted. The results were listed in Table 2.

### EXAMPLE 30

The same method as Example 1 was used except the feed volume ratio of N2:CO:methyl nitrite=20:40:40 was adopted. The results were listed in Table 2.

### EXAMPLE 31

The same method as Example 1 was used except the feed volume ratio of N2:CO:methyl nitrite=40:40:20 was adopted. The results were listed in Table 2.

### EXAMPLE 32

The same method as Example 1 was used except the feed volume ratio of N2:CO:methyl nitrite=50:40:10 was adopted. The results were listed in Table 2.

### EXAMPLE 33

The same method as Example 1 was used except the feed volume ratio of N2:CO:methyl nitrite=70:25:5 was adopted. The results were listed in Table 2.

### EXAMPLE 34

The same method as Example 1 was used except the residence time of 1 second was adopted. The results were listed in Table 2.

Provided was a monolithic catalyst for synthesizing an oxalate by carbon monoxide(CO) gas-phase coupling. Compared with the supported particulate catalyst, the monolithic catalyst exhibited excellent catalytic performance of 450 gDMO/L.h (see Example 13), which was high than the particulate catalyst with 409 gDMO/L.h (see Comparative Example 5). The highest space-time yield of oxalate can be achieved to be 920 gDMO/L.h through the modification of the reaction conditions. In addition, the absolute loading of noble metal in the structured catalyst per unit volume only account for 14% of that In particulate catalyst. It's obvious that monolithic catalyst saved more than 86% precious metal, significantly reducing the cost of the catalyst and the production cost of the oxalate. Meanwhile, the monolithic catalyst has neatly ranged parallel channels, larger porosity of the catalyst bed and lower resistance for the reaction stream flowing through the catalyst bed, accelerating the large-scale industrial application of the technology.

TABLE 1
The performance of the catalyst for synthesizing dialkyl oxalate from carbon
gaseous coupling

| | | | | | active | | | | | |
|---|---|---|---|---|---|---|---|---|---|---|
| | | | | | component | | | | | |
| | milling | washcoat | washcoat | Active | loading/ | | | Pd/ | CO | STY, |
| Catalyst | time/h | composition | loading/wt. % | component | wt. % | Additive | gPd/L | Fe | conversion/% | g/L · h |
| Example 1 | 16 | Al₂O₃ | 20 | Pd | 1 | Fe | 1.14 | 1.5 | 32 | 347 |
| Example 3 | 16 | Al₂O₃ | 20 | Pd | 1 | Fe | 1.14 | 1.5 | 32 | 339 |
| Example 4 | 4.5 | Al₂O₃ | 20 | Pd | 1 | Fe | 1.14 | 1.5 | 27 | 276 |
| Example 5 | 9 | Al₂O₃ | 20 | Pd | 1 | Fe | 1.14 | 1.5 | 29 | 308 |
| Example 6 | 36 | Al₂O₃ | 20 | Pd | 1 | Fe | 1.14 | 1.5 | 33 | 357 |
| Example 7 | 16 | Al₂O₃ | 5 | Pd | 1 | Fe | 0.29 | 2 | 28 | 292 |
| Example 8 | 16 | Al₂O₃ | 10 | Pd | 1 | Fe | 0.57 | 2 | 31 | 324 |
| Example 9 | 16 | Al₂O₃ | 30 | Pd | 1 | Fe | 1.71 | 3 | 39 | 390 |
| Example | — | SiO₂ | 20 | Pd | 1 | Fe | 1.14 | 1.5 | 31 | 321 |
| 10 | | | | | | | | | | |
| Example | 16 | TiO₂ | 20 | Pd | 1 | Fe | 1.14 | 1.5 | 29 | 300 |
| 11 | | | | | | | | | | |
| Example | 16 | ZrO₂ | 20 | Pd | 1 | Fe | 1.14 | 1.5 | 27 | 285 |
| 12 | | | | | | | | | | |
| Example | 16 | Al₂O₃—MgO | 20 | Pd | 1 | Fe | 1.14 | 1.5 | 42 | 450 |
| 13 | | | | | | | | | | |
| Example | 16 | Al₂O₃—MgO | 20 | Pd | 1 | Fe | 1.14 | 1.5 | 34 | 367 |
| 14 | | | | | | | | | | |
| Example | 16 | Al₂O₃ | 20 | Pd | 0.1 | Fe | 0.11 | 1.5 | 16 | 176 |
| 15 | | | | | | | | | | |
| Example | 16 | Al₂O₃ | 20 | Pd | 2 | Fe | 2.28 | 1.5 | 34 | 361 |
| 16 | | | | | | | | | | |
| Example | 16 | Al₂O₃ | 20 | Pd | 1 | Fe | 1.14 | 0.1 | 28 | 310 |
| 17 | | | | | | | | | | |
| Example | 16 | Al₂O₃ | 20 | Pd | 1 | Fe | 1.14 | 2.5 | 33 | 332 |
| 18 | | | | | | | | | | |
| Example | 16 | Al₂O₃ | 20 | Pd | 1 | Ni | — | 1 | 29 | 301 |
| 19 | | | | | | | | | | |
| Example | 16 | Al₂O₃ | 20 | Pd—Ir | 0.9 | Fe | — | 1.2 | 29 | 301 |
| 20 | | | | | | | | | | |
| Example | 16 | Al₂O₃ | 20 | Pd | 1 | Fe | 1.14 | 1.5 | 35 | 385 |
| 21 | | | | | | | | | | |
| Example | 16 | Al₂O₃ | 20 | Pd | 1 | Fe | 1.14 | 1.5 | 34 | 370 |
| 22 | | | | | | | | | | |
| Example | 16 | Al₂O₃ | 20 | Pd | 1 | Fe | 1.14 | 1.5 | 33 | 355 |
| 23 | | | | | | | | | | |
| Example | 16 | Al₂O₃ | 20 | Pd | 1 | Fe | 1.14 | 1 | 32 | 336 |
| 24 | | | | | | | | | | |
| Comparative | 16 | Al₂O₃ | 20 | Pd | 1 | Fe | 1.14 | 1.5 | 28 | 300 |
| example 2 | | | | | | | | | | |
| Comparative | — | Al₂O₃ | 20 | Pd | 1 | Fe | 1.14 | 1.5 | 19 | 220 |
| example 3 | | | | | | | | | | |

TABLE 2
The performance of the catalyst for synthesizing dialkyl oxalate from carbon
gaseous coupling

| | | | | | | Pd | | | |
|---|---|---|---|---|---|---|---|---|---|
| | Pressure/ | Tempur/ | N2:CO:AN | residence | Alkyl | loading/ | | CO | STYg/ |
| Catalyst | MPa | ° C. | (v/v/v) | time/s | nitrite | wt. % | gPd/L | conversion/% | L · h |
| Example | 0.1 | 120 | 40:40:20 | 1.2 | ethyl | 1 | 1.14 | 34 | 420 |
| 25 | | | | | nitrite | | | | |
| Example | 0.3 | 130 | 40:40:20 | 3 | methyl | 1 | 1.14 | 38 | 470 |
| 26 | | | | | nitrite | | | | |
| Example | 0.6 | 110 | 50:30:20 | 3.6 | methyl | 1 | 1.14 | 45 | 920 |
| 27 | | | | | nitrite | | | | |
| Example | 0.1 | 90 | 50:30:20 | 1.5 | methyl | 1 | 1.14 | 24 | 257 |
| 28 | | | | | nitrite | | | | |
| Example | 0.1 | 150 | 50:30:20 | 1.5 | methyl | 1 | 1.14 | 58 | 608 |
| 29 | | | | | nitrite | | | | |
| Example | 0.1 | 110 | 20:40:40 | 1.5 | methyl | 1 | 1.14 | 50 | 530 |
| 30 | | | | | nitrite | | | | |
| Example | 0.1 | 110 | 40:40:20 | 1.5 | methyl | 1 | 1.14 | 37 | 406 |
| 31 | | | | | nitrite | | | | |
| Example | 0.1 | 110 | 50:40:10 | 1.5 | methyl | 1 | 1.14 | 20 | 215 |
| 32 | | | | | nitrite | | | | |
| Example | 0.1 | 110 | 75:20:5 | 1 | methyl | 1 | 1.14 | 18 | 105 |
| 33 | | | | | nitrite | | | | |
| Example | 0.1 | 110 | 50:30:20 | 1 | methyl | 1 | 1.14 | 22 | 355 |
| 34 | | | | | nitrite | | | | |

While embodiment of the present invention have been illustrated and described, such disclosures should not he regarded as any limitation of the scope of our invention. The true scope of our invention is defined in the appended claims. Therefore, it is intended that the appended claims shall be construed to include both the preferred embodiment and all such variations and modifications as fall within the spirit and scope of the invention.

1-21. (canceled)

22: A monolithic structured catalyst for carbon monoxide gas-phase coupling to dialkyl oxalate comprising;
a honeycomb support having a coating of metal oxides;
active components including at least one precious metal impregnated onto the coating; and
additives selected from the group consisting of Fe, Co, Ni and mixtures thereof impregnated onto the coating.

23: The monolithic structured catalyst according to claim 22, wherein the metal oxide are selected from the group consisting of the following: Al2O3, SiO2, ZrO2 TiO2, Fe2O3, La2O3, CuO, ZnO, Cr2O3, GaO, BaO, CaO, MgO, MnO and mixtures thereof.

24: The monolithic structured catalyst according to claim 22, wherein the active ingredient was selected from the group consisting of Pt, Pd, Ir, Rh and mixtures thereof.

25: The monolithic structured catalyst according to claim 22 wherein the additives also includes Cu or Ce and mixtures thereof.

26: The monolithic structured catalyst according to claim 22, wherein the coating of metal oxides accounts for 5 to 50 wt. % of the honeycomb support; the active Ingredients account for 0.1 to 5 wt. % of the coating on the honeycomb support; the additives account for 0.03 to 10 wt. % of the coating on the honeycomb support; and the active ingredients having an atomic ratio to the additives of 0.01 to 5.

27: The monolithic structured catalyst according to claim 22, wherein the coating on the honeycomb support accounts for 5 to 5.0 wt. % of the honeycomb support; the active Ingredients account for 0.1 to 5 wt. % of the coating on the honeycomb support; the additives account for 0.3 to 10 wt. % of the coating on the honeycomb support; and the active ingredients having an atomic ratio to the additives of 0.1 to 5.

28: The monolithic structured catalyst according to claim 22, wherein coating on the honeycomb support accounts for 5 to 30 wt. % of the honeycomb support: the active ingredients account for 0.1 to 2 wt. % of the coating on the honeycomb support; the additives account for 0.3 to 6 wt. % of the coating on the honeycomb support; and the active ingredients having an atomic ratio to the additives of 0.1 to 3.

29: The monolithic structured catalyst according to claim 22, wherein the honeycomb support is ceramic.

30: The monolithic structured catalyst according to claim 22, wherein the honeycomb support is metal.

31: A method for preparing a monolithic structured catalyst for synthesizing an oxalate by carbon monoxide(CO) gas-phase coupling comprising the steps of:
mixing at least one of the group consisting of metal nitrate, hydroxide and oxide with dilate nitric acid, adjusting the pH of the mixture in the range of 1 to 4, ball milling the mixture between 1 and 48 hours to create a washcoat;
applying the washcoat on a honeycomb support use a dip-coating method;
drying the washcoated honeycomb support;
repeating the applying and drying steps until the desired amount of washcoat has been applied;
calcinating the washcoat through a heating process performed at 900-1300° C. for 1-12 hours;
impregnating the washcoat with active components and additives; and
treating the coated honeycomb support in H2 or CO atmosphere for 1-10 hours.

32: The method for preparing a monolithic structured catalyst according to claim 31, wherein the impregnated washcoat is treated in the 0.01-2M alkaline solution for 0.5-24 hours after drying.

33: A method for preparing a monolithic structured catalyst according to claim 32, wherein the alkaline solution was selected from the group consisting of NaOH, KOH, Na2CO3, K2CO3, NaHCO3, KHCO3 and mixtures thereof.

34: A method for preparing a monolithic structured catalyst according to claim 31, wherein the active ingredient is selected from the group consisting of palladium chloride, palladium bromide, chloride platinum and rhodium chloride, palladium nitrate, nitrate platinum acetate palladium, acetate rhodium platinum group metal salts and mixtures thereof.

35: A method for preparing a monolithic structured catalyst according to claim 31, wherein the additive is selected from the group consisting of ferric chloride, cobalt bromide, nitrate iron, nickel nitrate, iron phosphate, cobalt phosphate or cobalt acetate, nickel acetate and mixtures thereof.

36: A method for preparing a monolithic structured catalyst according to claim 31, wherein:
mixing at least one of the: group consisting of metal nitrate, hydroxide and oxide with dilute nitric acid of 1-15 wt. %, adjusting the pH of the mixture in the range of 1 to 4, ball milling the mixture between 3 and 20 hours to create a washcoat;
applying the washcoat on a honeycomb support use a dip-coating method;
drying the washcoated honeycomb support at 70-130° C. for 2-4 hours*
repeating the applying and drying steps until the washcoat accounts for 5-50 wt. % of honeycomb support has been applied;
calcinating the washcoat through a heating process performed at 900-1200° C. for 1-12 hours:
impregnating the washcoat with active components and additives for 3 minutes to 12 hours;
drying the impregnated honeycomb support at 70-130° C. for 1-12 hours; and
treating the coated honeycomb support in H2 or CO atmosphere for 1-10 hours.

37: A method for synthesizing oxalate by carbon monoxide (CO) gaseous-phase coupling for using with a monolithic structured catalyst comprising the steps of:
filling a catalyst bed located within a fixed bed reactor with a monolithic structured monolithic catalyst supporting noble metal wherein the reaction pressure was 0.1-2 MPa, the reaction temperature was 80-200° C. and N2 was used as carrier gas; and
introducing CO and gasified alkyl nitrite into the reactor to react on the monolithic structured catalyst to produce dialkyl oxalate, wherein the volume ratio of N2:CO:Alkyl nitrite was 20-80:5-60:10-40, and the retention time was 0.5-10 s.

38: A method according to claim 37, wherein the reaction pressure was 0.1-2 MPa, the reaction temperature was 90-150° C.

39: A method according to claim 37, wherein the volume ratio of N2:CO:Alkyl nitrite was 20-80:5-60:10-40, and the retention time was 1-10 s.

40: A method according to claim 37, wherein the volume ratio of N2:CO:Alkyl nitrite was 20-80:5-60:5-10.

41: A method according to claim 37, wherein the product dialkyl oxalate is selected from the group consisting of diethyl oxalate, dimethyl oxalate and mixtures thereof; and the alkyl nitrite is selected from the group consisting of ethyl nitrite methyl nitrite and mixtures thereof.
